(19) United States
(12) Patent Application Publication
(10) Pub. No.: US 2015/0165148 A1
(43) Pub. Date: Jun. 18, 2015

(54) Endotracheal Tube Insertion Device

(71) Applicant: Catherine Susan Kozlowski, Albuquerque, NM (US)

(72) Inventor: Catherine Susan Kozlowski, Albuquerque, NM (US)

(21) Appl. No.: 14/531,960

(22) Filed: Nov. 3, 2014

Related U.S. Application Data
(60) Provisional application No. 61/901,369, filed on Nov. 7, 2013.

(51) Int. Cl. A61M 16/04

(52) U.S. Cl. CPC A61M 16/0497; A61M 16/0434; A61M 2209/06

(57) ABSTRACT

A device is presented to aid in endotracheal intubation which eases insertion of the endotracheal tube. The insertion device is preferably pre-assembled with a standard endotracheal tube, and the assembly is inserted into the patient's airway, stopping at the esophagus. The endotracheal tube is then advanced to its usual position, and then the insertion device is removed. Easy removal of the insertion device is facilitated by direct access to the endotracheal tube along the anterior side of the insertion device.

### REFERENCES CITED

U.S. PATENT DOCUMENTS

| | | |
|---|---|---|
| 4,509,514 | April 1985 | Bain |
| 5,174,283 | December 1992 | Paker |
| 5,339,805 | August 1994 | Paker |
| 6,079,409 | June 2000 | Bain |
| 7,174,889 | June 2004 | Boedeker et al. |

### OTHER PUBLICATIONS

Hussain and Redmond, Are pre-hospital deaths from accidental injury preventable?, B M J 1994; 308: 1077

Gerstein et al. Lingual Ischemia from Prolonged Insertion of a Fastrach Laryngeal Mask Airway, The Western Journal of Emergency Medicine, 2011 February; 12(1): 124-127

Eastridge et al. Death on the battlefield (2001-2011): Implications for the future of combat casualty care, Journal of Trauma and Acute Care Surgery: December 2012, Volume 73, Issue 6 p S431-S437.

Young, The intubating laryngeal-mask airway may be an ideal device for airway control in the rural trauma patient, Am Journal of Emergency Medecine, 2003 January; 21(1):80-5

### BACKGROUND OF THE INVENTION

In medical practice, it is often necessary to secure the airway of a patient. A 1994 study by Hussain and Redmond, (BMJ 1994; 308: 1077) indicated that up to 39% of trauma deaths may have been preventable with more expedient airway management. In their 2012 paper Death on the battlefield (2001-2011): Implications for the future of combat casualty care, Eastridge et al. indicate that airway obstruction is the second most common physiologic cause of battlefield mortality, after hemorrhage.

In current practice, securing the airway is done primarily through endotracheal (ET) intubation, or by using a device known as a laryngeal mask airway. Each of these methods has different advantages and disadvantages. With endotracheal tubes, the airway is relatively secure, with a seal around the endotracheal tube to the trachea. This seal can prevent blood, gastric contents and other secretions from being aspirated. The disadvantage of an endotracheal tube is the relatively difficult insertion. Insertion via direct laryngoscopy requires manipulation of the patients head into an optimum viewing position and the use of a laryngoscope to visually identify the airway. The endotracheal tube is then inserted manually with the airway thus visualized. The operation of the laryngoscope requires significant skill, and can lead to patient injury if the insertion is done improperly or if the patient has a compromised spinal column or other injury which can be damaged during manipulation of the head.

Conversely, the laryngeal mask airway has the advantage of easy installation, usually requiring significantly less manipulation of the head position of the patient. The laryngeal mask airway also requires significantly less skill to install than the traditional endotracheal tube owing to the anatomical shape of the laryngeal mask. It is thus more suitable to persons who perform intubation either less frequently, or who require more expedient intubation, such as combat medics or emergency paramedics. The significant disadvantage of the laryngeal mask is that it does not protect the airway as well as traditional endotracheal intubation from the aspiration of blood, gastric contents or other secretions, and is less able to support pressurized airway. support.

It is desirous to develop techniques and equipment to perform intubation with the ease of insertion of the laryngeal mask airway, and the secure airway management of the endotracheal tube. To this end, the intubating laryngeal mask airway (ILMA) has been developed (see for example U.S. Pat. No. 6,079,409). While the laryngeal mask devices typically have a flexible airway tube, in the intubating laryngeal mask devices, the airway tube is replaced with a rigid thin wall tube to allow for endotracheal tube insertion. The rigidity of the airway tube can, in some cases, lead to injury or inflammation of the patient (for example, see Gerstein et al. Lingual Ischemia from Prolonged Insertion of a Fastrach Laryngeal Mask Airway). An additional disadvantage of the intubating laryngeal mask is the relative difficulty of transitioning the device from operating as a laryngeal mask to traditional intubation. To accomplish this transition, the endotracheal tube is advanced along the lumen of the intubating laryngeal mask until it reaches the desired position.

The endotracheal tube's cuff is expanded in the usual manner, and then the intubating laryngeal mask is withdrawn. To prevent the endotracheal tube from being dislodged during removal of the intubating laryngeal mask, a push rod or stylette must be used to hold the endotracheal tube in position since the body of the endotracheal tube is not accessible by hand. In usual practice, ventilation of the patient is not possible during removal of the intubating laryngeal mask. This has been addressed somewhat via U.S. Pat. No. 7,174,889, but this solution is inadequate owing to still requiring an additional device to press on the endotracheal tube, leading to potential complications.

Parker (U.S. Pat. Nos. 5,339,805 and 5,174,283) has disclosed blind orolaryngeal and oroesophageal guiding and aiming devices which purport to accomplish goals similar to those stated here. However, investigation of the Parker device shows that it shares the chief difficulty of the intubating laryngeal mask devices, in that removal of the device after successful intubation is problematic. Once the endotracheal tube is inserted into the final position, direct access to the endotracheal tube is limited, preventing stabilization of the endotracheal tube during removal of the guiding device. This is because the Parker device is closed along both the anterior and posterior edges. Also, similar to the intubating laryngeal mask device, the design of the Parker device would require relatively rigid materials to successfully provide guidance of the endotracheal tube down the entire length of the guiding device.

### OBJECTS OF THE INVENTION

There is an established need to develop an endotracheal insertion device which is easy to use, allows for fast and accurate intubation, and can be preferentially used blindly (i.e. without direct visual confirmation of the airway). To this end, several devices have been developed which attempt to solve these issues, but all of them fail to provide easy, direct intubation using an endotracheal tube with relatively easy removal of the insertion device. Further, in each case of the prior art, the insertion device is inserted into the airway separate from the endotracheal tube. This increases the length of time the patient is without ventilation support, which is undesirable.

Preferably, a device would be developed which allows for an insertion device and an endotracheal tube be inserted into the patient's airway simultaneously. This would allow the insertion device to be flexible in construction as the tip of the endotracheal tube is not required to be guided along the entire length of the insertion device. It is further desired that the insertion device be separable from the endotracheal tube such that direct manual control of the endotracheal tube is possible during removal of the insertion device.

### SUMMARY OF THE INVENTION

An object of the present invention is to provide an endotracheal intubation insertion device which facilitates fast, accurate, blind access to the correct positioning of the endotracheal tube.

According to the present invention, the insertion device will have an anatomically shaped positioning feature at the distal end, a flexible longitudinal portion, and features for holding the endotracheal tube radially in place on both ends of the longitudinal portion. Airway management is provided through an endotracheal tube which is not permanently attached to the insertion device, and is free to translate in the longitudinal direction. The longitudinal portion has means along the (preferably) anterior side to permit direct manual access to the endotracheal tube to facilitate advancement of the endotracheal tube and removal of the insertion device.

In one embodiment, the insertion device will have a shape similar to the laryngeal mask airways of prior art, with an inflatable cuff providing a sealing means with the airway tube. The longitudinal portion of the present invention replaces the airway tube portion of the standard laryngeal mask airway.

According to the present invention, the endotracheal tube is placed within the insertion device, the two objects thus becoming an assembly. The endotracheal tube is held in radial orientation with respect to the insertion device in both the distal and proximal end of the longitudinal portion. The material of the endotracheal tube will thus add some rigidity to the flexible handle portion to aid in insertion, with the assembly remaining substantially flexible in the preferred embodiment. It is recognized that specific design concerns may require a rigid or semi-rigid longitudinal portion for specific applications (for example, non-human species), so the present invention is not thus limited to a flexible longitudinal portion.

The assembly of the insertion device and endotracheal tube is advanced through the airway until the stopper portion lodges in the esophagus. The endotracheal tube is then advanced along the longitudinal portion of the insertion device until it is in the usual final position. The design of the longitudinal portion is such that when the endotracheal tube is advanced to its usual final position, the proximal end of the endotracheal tube will be free from radial confinement with respect to the insertion device.

The endotracheal tube cuff is then expanded in the usual way, and then the insertion device is removed along the length of the now properly installed endotracheal tube. In this way, it is quite similar to the intubating laryngeal mask airway, except that direct manual access is now granted to the body of the endotracheal tube (by means of the anterior cutout) during removal of the installation device, thus obviating the need for the rod or stylette as used with intubating laryngeal mask devices.

In a second embodiment, the inflatable cuff portion of the stopper is replaced with a non-inflatable positioning device with an anatomical shape. Such stopper may be made of elastomeric materials or similar.

In a third embodiment, the proximal end of the longitudinal portion which positions the endotracheal tube radially will include an axial split to allow easier separation of the endotracheal tube from the longitudinal portion.

In a fourth embodiment, the distal end of the longitudinal portion which positions the endotracheal tube radially will include an axial split to allow easier separation of the endotracheal tube from the longitudinal portion. This may be in addition to or instead of the split disclosed at the proximal end.

In a fifth embodiment, the axial splits identified in the third and fourth embodiments may be alternately replaced with a predominantly axial section of significantly reduced material thickness to allow the axial split to be added post-insertion by mechanical manipulation (e.g. tearing) of the device.

In a sixth embodiment, sealing means are provided at the distal end between the endotracheal tube and the radial positioning feature. In this way, the device may be used in the same fashion as a laryngeal mask airway. This embodiment is accompanied by a stopper in the form of an inflatable cuff or other means of sealing the stopper with the airway.

In a seventh embodiment, the anatomically shaped stopper includes a bypass feature (such as a cut out) which will guide the endotracheal tube into the correct position in the event that the insertion device is mistakenly pushed into the trachea. Proximal to the guiding feature is a means to prevent the endotracheal tube from passing through the bypass feature when the stopper is properly inserted into the esophagus. If the anatomically shaped stopper is placed in the trachea, the stopper will be deflected due to interference with the anatomy in a way which is different than when the stopper is engaged in the esophagus. The deflection of the stopper when engaged in the trachea will allow the bypass feature to be presented to the endotracheal tube. In this way, the endotracheal tube is not blocked from passing into the trachea even in the event that the stopper is incorrectly inserted into the trachea.

### BRIEF DESCRIPTION OF THE DRAWINGS

FIG. 1 is a view of the insertion device from the anterior side, without the endotracheal tube installed.

FIG. 2 is a lateral view of the insertion device with the endotracheal tube preinstalled, shown with various cut away sections.

FIG. 3 is an alternate construction of the longitudinal portion with several windows to allow access to the endotracheal tube.

FIG. 4 is an anatomical cut away drawing showing the sub-assembled insertion device and endotracheal tube inserted such that the anatomically shaped stopper has correctly seated in the esophagus. The endotracheal tube is shown in its original preinstalled position.

FIG. 5 is a view from the anterior side of the insertion device assembled with the endotracheal tube. The embodiment shown includes predominately axial splits at both the distal and proximal ends. The endotracheal tube is shown in its preinstalled position.

FIG. 6 is an anatomical cut away drawing showing the sub-assembled insertion device and endotracheal tube inserted such that the anatomically shaped stopper has correctly seated in the esophagus. In this view, the endotracheal tube has been advanced to its final position.

FIG. 7 is an exploded cut away view of the distal end of the subassembly, showing sealing means between the insertion device and the endotracheal tube.

FIG. 8 is an exploded view of the anatomically shaped stopper, showing a relief feature to allow the endotracheal tube to pass into the trachea in the event that the stopper is mistakenly inserted into the trachea as opposed to the esophagus.

### DETAILED DESCRIPTION OF THE PREFERRED EMBODIMENTS

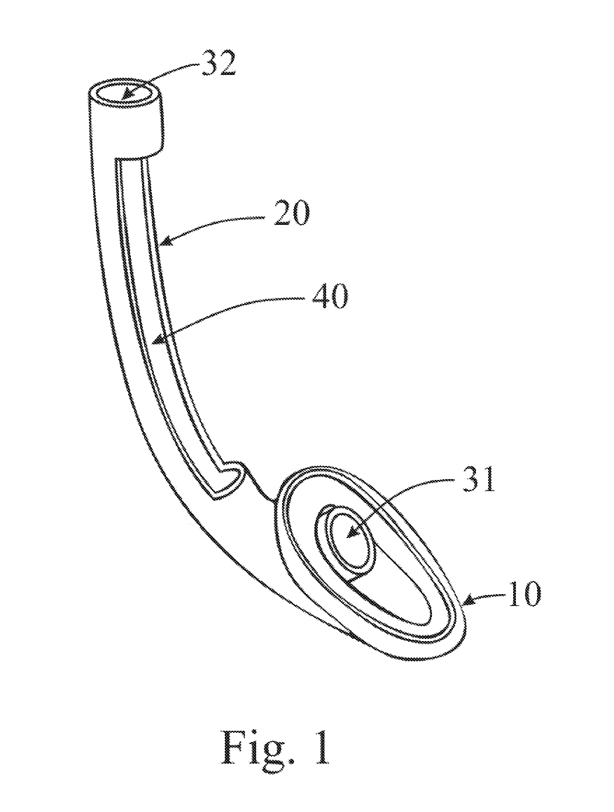

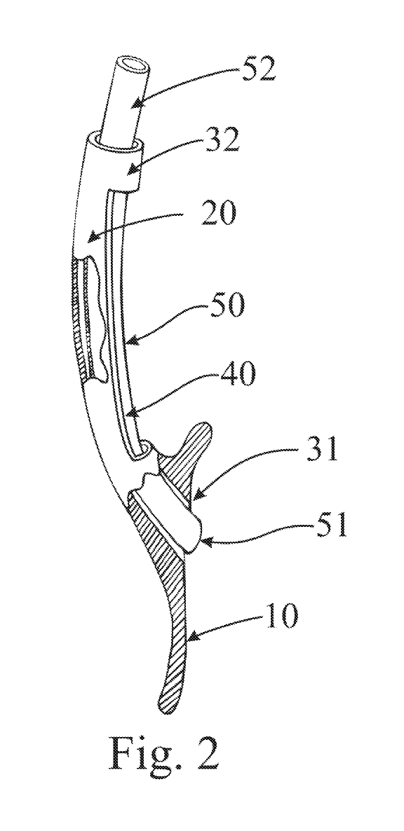

With reference to FIGS. 1 and 2, an insertion device for endotracheal tubes comprises an anatomically shaped stopper (10), a longitudinal portion (20), a distal annulus (31), a proximal annulus (32), and means (40) to facilitate access to an endotracheal tube positioned between the annuli (31 and 32). It is understood that the anatomically shaped stopper (10) may be an inflatable feature, similar to present laryngeal masks, or alternately an appropriately designed feature made of elastomeric material or similar. FIG. 2 further demonstrates the position of the endotracheal tube (50) within the insertion device. The endotracheal tube (50) has a distal end (51) and proximal end (52). While the endotracheal tube (50) is shown without the usual inflatable cuff, it is to be understood that the preferred embodiment the endotracheal tube will include the inflatable cuff.

The longitudinal portion (20) is preferably flexible, with it understood that the degree of flexibility dictated by specific design criteria. The flexibility of the longitudinal portion (20) will facilitate easy insertion without undue trauma on the patient's anatomy. The longitudinal portion (20) is constructed in the preferred embodiment as a substantially tubular shape which fully encircles the endotracheal tube (50) at both the distal and proximal end, forming the annuli (31) and (32), but does not fully encircle the endotracheal tube (50) over at least some portion, forming the means (40) to access the endotracheal tube (50).

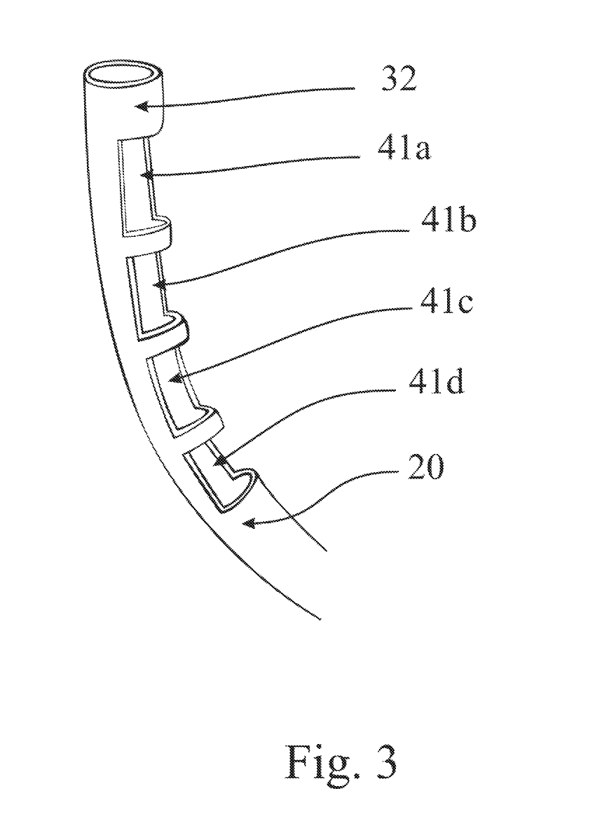

In the preferred embodiment, the means (40) to facilitate access to the endotracheal tube is comprised of one or more window shaped aperture(s) along the anterior edge of the substantially tube shaped longitudinal portion (20), as shown in FIGS. 1 and 2. FIGS. 1 and 2 as drawn show a single long window extending the majority of the length of the longitudinal portion (20), but it is understood that the long window may be by a multitude of smaller windows (41a through 41d) arranged along the length of the longitudinal portion (20), as demonstrated in FIG. 3.

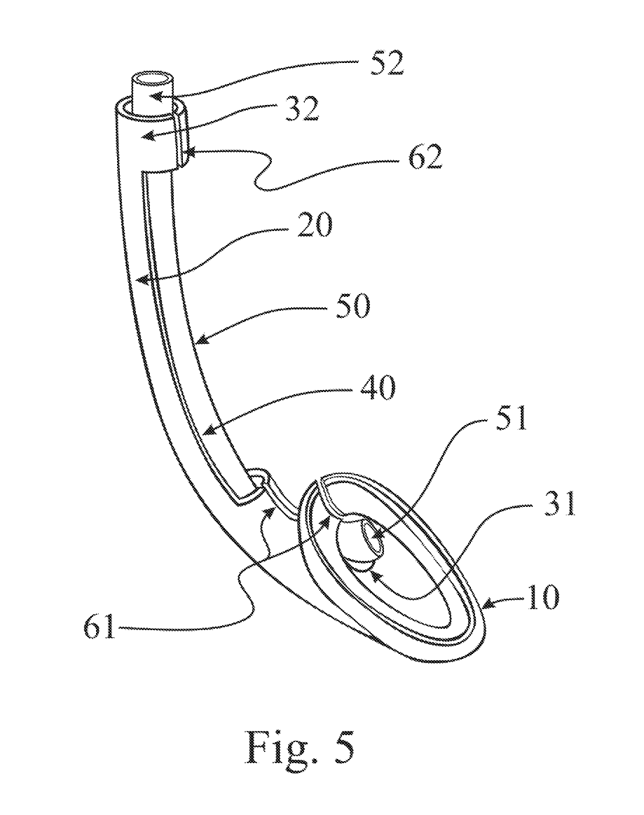

A preferred embodiment includes predominantly axial splits at either one or both of the annuli (31 and 32) to allow disengagement of the endotracheal tube (50). These splits are shown in FIG. 5, with in both the distal (61) and proximal (62) slit shown, but it is understood that the design may include neither axial slit, either one alone, or both together.

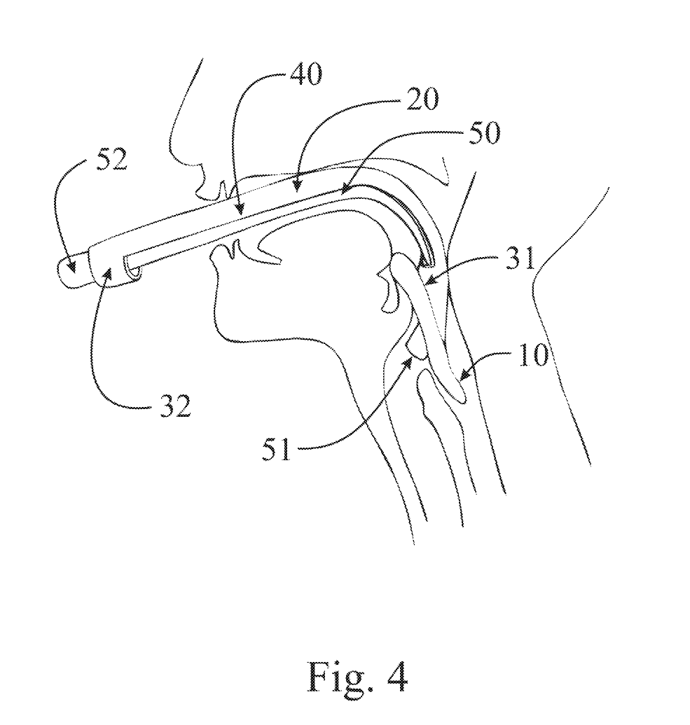

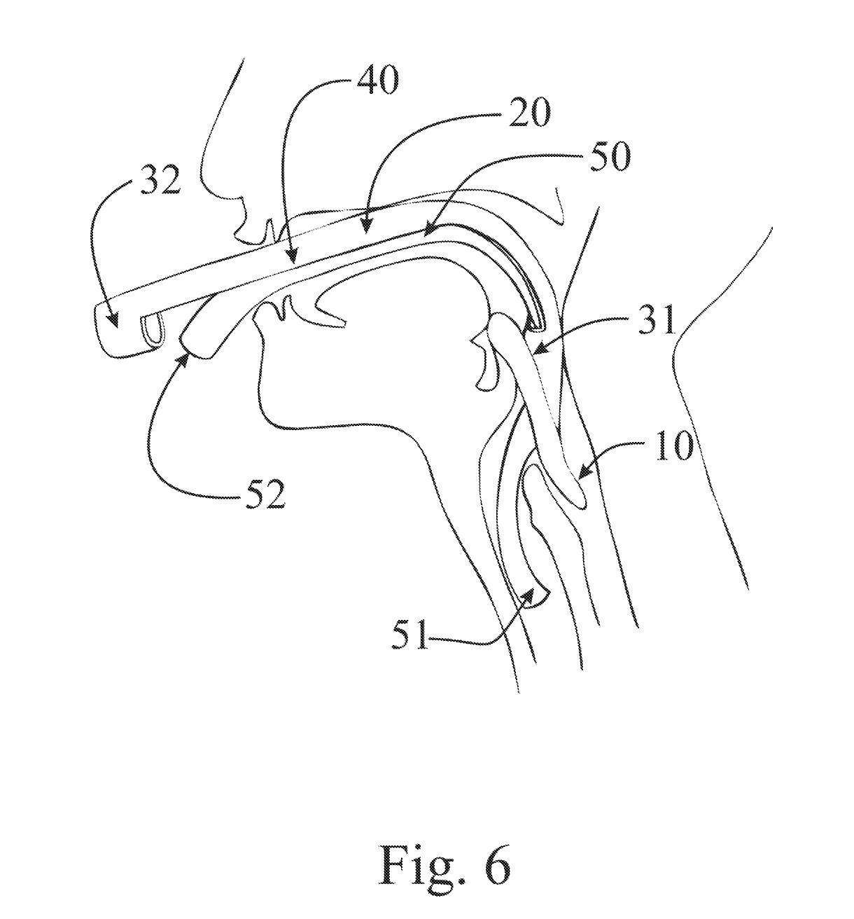

In normal usage, the endotracheal tube (50) is pre-assembled into the insertion device, with the distal end (51) engaged within the distal annulus (31), and the proximal end (52) engaged within the proximal annulus (32), as shown in FIGS. 2, 4 and 5. The subassembly is inserted through the patient's mouth, guided along the hard and soft palate, and advanced along the oropharynx. The anatomically shaped stopper (10) is shaped such that the distal tip of the stopper (10) is guided into the esophagus during normal insertion, preventing further advancement of the assembly, as demonstrated in FIG. 4. The endotracheal tube (50) is then advanced such that the distal end (51) is guided along the trachea to the usual position, as shown in FIG. 6. As can be seen in FIG. 6, the proximal end (52) of the endotracheal tube (50) is free from constraint by the proximal annulus (32) when in the advanced position thus shown. This is an important feature, and forms a significant difference with the prior art. In the case of prior art, the endotracheal tube's (50) proximal end (52) remains radially constrained by the annulus of the insertion device. With the proximal end of the endotracheal tube (52) thus unconstrained, it may be grasped directly by hand. This allows the endotracheal tube (50) to be maintained in the correct position during removal of the insertion device.

The endotracheal tube (50) would have its cuff (not shown) inflated in the usual manner, and ventilation of the patient could proceed. If desired, the insertion device may be removed at this point. In embodiments with the predominantly axial splits (61 and 62) at the distal (31) and proximal (32) annuli, the installation device may be removed without the interruption of the ventilation process. This is in sharp contrast to previous designs wherein the ventilation process must be interrupted at some point in the extraction process.

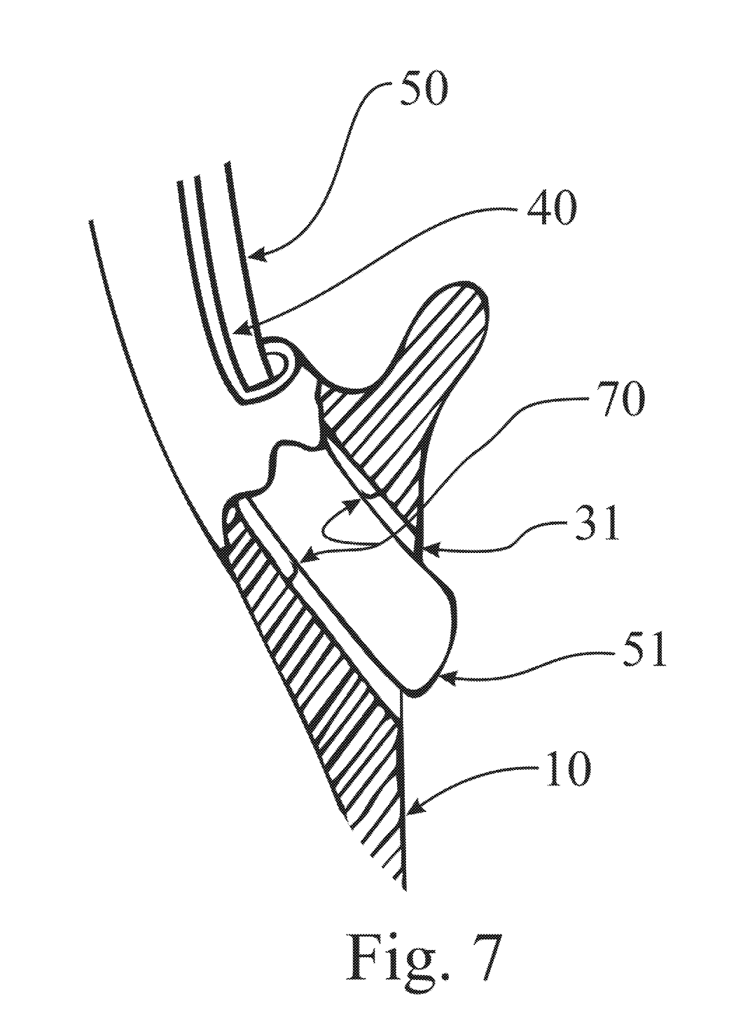

In the situation where it is desirous to use the sub-assembled insertion device and endotracheal tube (50) in the manner which is usual for a laryngeal mask airway, it is required to provide sealing means (70) between the distal aperture (31) and the distal end of the endotracheal tube (51), as shown in FIG. 7. This design configuration may eliminate the ability to include the distal slit (61) in the distal aperture (31). Although the sealing means (70) is shown in a simplified manner, it is understood that the sealing means (70) may take on any variety of shape which is usual for sealing of a cylinder within an annulus, including O-rings, protrusions which are arcuate in shape, protrusions with fingers extending from the annulus to the endotracheal tube, protrusions which are triangular, square or round in cross section, etc.

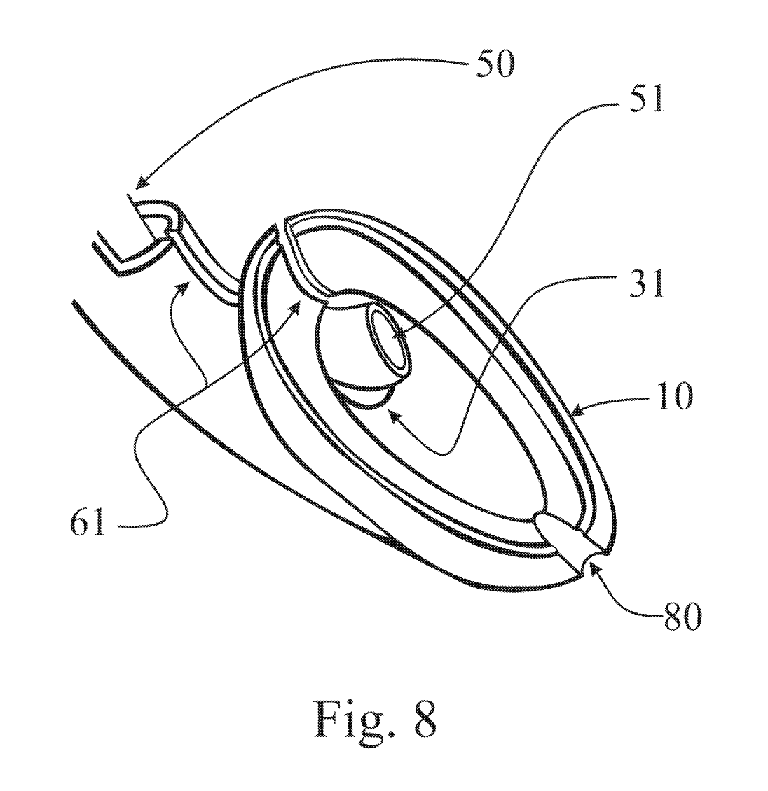

In practice, if the insertion device is advanced along the oropharynx very quickly, such as would be done in a trauma situation, or by an inexperienced practitioner, it is known from prior art that on occasion, the stopper (10) may become mistakenly advanced into the patient's trachea. In this circumstance, the distal tip of the stopper (10) may engage the anterior surface of the trachea and prevent the advancement of the endotracheal tube (50). In some instances, it will be desirable to include a feature (80) to allow the endotracheal tube (50) to advance along the trachea even with the stopper (10) lodged within the trachea. An example of such a feature (80) is shown in FIG. 8. It should be understood from FIG. 8 that the feature (80) is shown in a simplified manner, but may be brought to practice in a more complex shape.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description.

What is claimed is:

1. An endotracheal tube insertion device comprising:
a. an anatomically formed stopper portion at the distal end which causes the insertion device to stop at or near the top of the esophagus during insertion.
b. a longitudinal portion to provide means for advancement of the insertion device along an airway.
c. a pair of annuli which provide radial positioning of an endotracheal tube; a proximal annulus and distal annulus. The distal annulus is positioned near (but proximal to) the stopper end, and provides directional support such that the endotracheal tube advances in the direction of the trachea. The proximal annulus is positioned in the longitudinal portion such that it supports the endotracheal tube during insertion of the assembly into the airway, but loses communication with the endotracheal tube once the endotracheal tube is advanced to its final position. Both annuli are sized such that axial motion of the endotracheal tube is smooth and unimpeded.
d. means on the longitudinal portion of the insertion device facilitating direct manipulation of the endotracheal tube during extraction of the insertion device.

2. A device according to claim 1 where the means to facilitate direct manipulation of the endotracheal tube is located on the anterior side of the longitudinal portion.

3. A device according to claim 1 where the longitudinal portion is made of a tube with one or more window shaped cutouts on the anterior side to allow direct access to the installed endotracheal tube.

4. A device according to claim 1 where the anterior side access to the endotracheal tube is achieved by means of an axial slit along the length of the longitudinal portion of the insertion device.

5. A device according to claim 1 where the longitudinal portion is flexible.

6. A device according to claim 1 where the longitudinal portion is rigid or semi-rigid.

7. A device according to claim 1 where the anatomically formed stopper is comprised of an inflatable cuff.

8. A device according to claim 1 where the anatomically formed stopper is comprised of a non-inflatable anatomically shaped stopper.

9. A device according to claim 1 where the longitudinal portion partially encircles the endotracheal tube over some portion and fully encircles the endotracheal tube over other portions.

10. A device according to claim 1 where either one or both annuli have a predominantly axial split to allow disengagement of the endotracheal tube.

11. A device according to claim 10 where the predominantly axial split is formed by means of tearing the annulus along a pre-formed thin wall section during the intubation procedure.

12. A device according to claim 7 where the distal annulus provides sealing means with the endotracheal tube.

13. A device according to the claim 1 where the longitudinal portion includes a handle feature.

14. A device according to the claim 1 where the anatomically shaped stopper includes a relief feature to provide direction to the advancement of the endotracheal tube in the circumstance that the installation device is mistakenly pushed into the trachea as opposed to the esophagus.

15. A device according to claim 1 where the insertion device is pre-packaged with an endotracheal tube pre-positioned within the insertion device, either with or without lubricating fluid pre-applied.
